(19) United States
(12) Patent Application Publication
(10) Pub. No.: US 2011/0087312 A1
(43) Pub. Date: Apr. 14, 2011

(54) Method for Treatment of Diabetes and Prediabetes with Low-Level Laser Therapy

(75) Inventors: Charles SHANKS, McKinney, TX (US); Ryan MALONEY, Gilbert, AZ (US); Steven C. SHANKS, McKinney, TX (US); Kevin B. TUCEK, McKinney, TX (US); Rodrigo NEIRA, Red Deer (CA)

(73) Assignee: ERCHONIA CORPORATIN, McKinney, TX (US)

(21) Appl. No.: 12/971,362

(22) Filed: Dec. 17, 2010

Related U.S. Application Data
(63) Continuation-in-part of application No. 12/554,673, filed on Sep. 4, 2009.
Application No. 12/554,673 is a continuation-in-part of application No. 11/409,408, filed on Apr. 20, 2006.
Application No. 11/409,408 is a continuation-in-part of application No. 10/772,738, filed on Feb. 4, 2004, now Pat. No. 7,118,588.
Application No. 10/772,738 is a continuation-in-part of application No. 10/976,581, filed on Oct. 29, 2004.
Application No. 10/772,738 is a continuation-in-part of application No. 09/932,907, filed on Aug. 20, 2001, now Pat. No. 6,746,473.
Application No. 10/976,581 is a continuation-in-part of application No. 09/932,907, filed on Aug. 20, 2001, now Pat. No. 6,746,473.
Application No. 09/932,907 is a continuation-in-part of application No. 12/870,002, filed on Aug. 27, 2010.
Application No. 12/870,002 is a continuation-in-part of application No. 10/976,581, filed on Oct. 29, 2004.
(60) Provisional application No. 61/287,551, filed on Dec. 17, 2009.
(60) Provisional application No. 60/273,282, filed on Mar. 2, 2001.

(51) Int. Cl. A61N 5/06

(52) U.S. Cl. 607/89

(57) ABSTRACT

Low-level laser therapy is applied externally through the skin of the patient to targeted areas of a of a patient's body to treat diabetes or prediabetes. A patient is first diagnosed with diabetes or prediabetes, preferably by blood draws that measure insulin, A1C, or glucose levels. A therapeutic amount of laser energy to apply is determined from a patient's biological factors such as body mass index. The therapeutic amount of laser energy is applied one or more times over a two week time period. The patient is then retested for diabetes or prediabetes. If necessary, a new therapeutic amount of laser energy is determined according to any changes in the patient's diabetes or prediabetes diagnosis and any changes in the patient's biological factors. Adjusted treatments with low-level laser energy continue until the diabetes or prediabetes diagnosis is removed.

### CROSS REFERENCE TO RELATED APPLICATIONS

This application claims the benefit of U.S. Provisional Application No. 61/287,551 filed Dec. 17, 2009. This application also is a continuation-in-part of and claims the benefit of co-pending U.S. patent application Ser. No. 12/554,673 filed Sep. 4, 2009, which claims the benefit of co-pending U.S. patent application Ser. No. 11/409,408 filed Apr. 20, 2006, which claims the benefit of co-pending U.S. patent applications Ser. No. 10/772,738 filed Feb. 4, 2004, and U.S. patent application Ser. No. 10/976,581 filed Oct. 29, 2004, both of which claim the benefit of U.S. patent application Ser. No. 09/932,907 filed Aug. 20, 2001, now U.S. Pat. No. 6,746,473, which claims the benefit of U.S. Provisional Application No. 60/273,282 filed Mar. 2, 2001. This application also is a continuation-in-part of and claims the benefit of co-pending U.S. patent application Ser. No. 12/870,002 filed Aug. 27, 2010 which claims the benefit of provisional application No. 61/237,795, filed Aug. 28, 2009 and is a continuation-in-part-of U.S. application Ser. No. 10/976,581 filed Oct. 29, 2004.

### FIELD OF INVENTION

This invention relates to a method for treating diabetes or prediabetes by external means. In particular, this invention relates to the application of laser energy to targeted external regions of a patient's body to reduce a patient's dependency on insulin and to slow the progression of diabetes.

### BACKGROUND

Diabetes mellitus, often just called diabetes, is a condition in which a person's body does not produce enough, or does not properly respond to, insulin. Insulin is a hormone produced in the pancreas that enables cells to absorb glucose to turn it into energy. When insulin production is insufficient or when the body does not properly respond to insulin, glucose accumulates in the blood, which can lead to various complications. While there are several forms of diabetes, three forms are the most recognized: type I diabetes, type II diabetes, and gestational diabetes. Additionally, prediabetes is recognized as preceding diabetes and exists when blood glucose levels that are higher than normal but not yet high enough to be diagnosed as diabetes.

Type I diabetes, which affects about 5-10 percent of Americans diagnosed with diabetes, is a metabolic disorder that is caused by destruction of the insulin-producing beta cells in the pancreas which leads to insulin deficiency and high levels of glucose in plasma. The onset of type I diabetes generally results from an autoimmune etiology; however, idiopathic causes of beta cell destruction can occur for type I. Type 1 diabetes can affect children or adults, but was traditionally termed “juvenile diabetes” because it represents a majority of the diabetes cases in children.

Type II diabetes is similar to other forms of diabetes as it presents with high levels of plasma glucose and is correlated with metabolic abnormalities. Generally, type II diabetes is characterized by insulin resistance, which also may be combined with reduced insulin secretion. The onset of type II diabetes is often gradual, and in the early stages of type II diabetes, the predominant abnormality is reduced insulin sensitivity. As type II diabetes progresses, the ability of the pancreas to secrete insulin is affected. Type II diabetes is the most common type of diabetes.

Gestational diabetes occurs in pregnant women who have not previously been diagnosed with diabetes but who have high glucose levels during pregnancy. Gestational diabetes affects about 4% of all pregnant women and may precede development of type II diabetes.

As the rate of obesity has increased in the United States, the prevalence of diabetes has increased as well. It has been reported that approximately 19 million Americans suffer from type II diabetes. In 1990, only 4.9% of individuals 18 years of age or older in the United States reported having diabetes. In 2001, however, 7.9% reported having diabetes. Patients considered obese saw the greatest prevalence of diabetes, with 14.9% of persons having a 35-39.9 kg/m² BMI and 25.6% of persons having a >40 kg/m² BMI suffering from diabetes. It has been predicted that an individual born in the year 2000 has 36% chance of developing diabetes in his or her lifetime.

Type II diabetes has been found to possess inheritable aspects which can be greatly impacted by external environmental factors. The underlying etiologies of type II diabetes include deficiencies in insulin-producing beta cells; altered response to insulin by muscle, adipose, and liver cells; and abnormalities in the regulating mechanisms responsible for controlling carbohydrate and lipid metabolism following ingestion of food. Modulation in insulin-sensitivity is affected by environmental factors and behaviors, mostly a sedentary lifestyle and obesity. The cellular mechanisms that contribute to modulation of muscle and adipose cell sensitivity to insulin are complex and are not well understood. It is believed that altering insulin signaling pathways, increasing the amount of intracellular fat, and elevating levels of free fatty acids and other adipose tissue products can impact insulin-sensitivity.

A strong relationship between excessive visceral adipose tissue and insulin-insensitivity has been established and continues to be extensively studied by diabetes researchers. The adipocyte is an endocrine organ cell responsible for the synthesis of bioactive peptides which participate in autocrine, paracrine, and endocrine pathways. Furthermore, adipose tissue is composed of a vibrant collection of cells including fibroblast, pre-adipocytes, immune cells, and a variety of other cells all embedded within the framework of connective tissue. It is believed that several adipose-derived bioactive substances are released through a coordinated fashion with other cells located in adipose tissue utilizing paracrine or autocrine pathways.

The diverse collection of cytokines and bioactive molecules (adipokines) released by adipose tissue have been shown to modulate lipid metabolism and homeostasis, energy consumption and expenditure, immunity, insulin sensitivity, and blood pressure. Directly associated with enlarged fat mass is the chronic disease diabetes. Adiponectin, a hormone solely produced by adipocytes, has demonstrated insulin sensitive effects promoting anti-diabetic characteristics. As a plasma protein, adiponectin has been reported to regulate insulin sensitivity via the activation of AMPK and reduction of mTOR/S6 kinase activity consequentially reducing insulin receptor substrate 1 inhibitory serine phosphorylation in several tissues. Similar to leptin, synthesis of adiponectin is tightly coupled with adipose tissue fat mass, demonstrating a negative relationship with larger masses. Individuals who are classified as obese display a lower plasma adiponectin concentration when compared to non-obese groups. Furthermore, a direct correlation between low adiponectin levels and the onset of type-2 diabetes has been reported. Adiponectin modulation is reflective of the deleterious outcome that manifest when the adipocyte accumulates tremendous volume. Lean fat mass is linked with the synthesis of beneficial hormones like adiponectin which promotes insulin sensitivity thus diabetes prevention. Reducing the fat mass is an important outcome in order to resolve related comorbidities such as diabetes.

Moreover, the production of tumor necrosis factor-α (TNF-α) and adipokine resistin, which has been shown to reduce concentrations of adipose cell hormone adiponectin, have been found to be elevated in patients with type II diabetes and have been shown to modulate insulin-sensitivity. In patients with and without diabetes, as weight and adiposity increase, beta cells compensate for the resulting decline in insulin sensitivity by increasing the insulin output, and the body's inability to adapt results in hyperglycemia.

If not properly controlled or stabilized, a hyperglycemic state has been associated with comorbidities including cardiovascular disease, vision impairment, various forms of neuropathy and cognitive impairment, stroke, and peripheral vascular disease. The common therapeutic approach, in addition to major modifications in an individual's dietary nutrition and physical activity, includes the use of antihyperglycemic drugs and insulin. Since the disease is chronic and progressive, it is imperative that a long-term strategy is constructed for each individual patient. Further, no treatment is unable to reverse the progression, and thus the modification of drug usage and the introduction of new or perhaps multiple agents are necessary to preserve patient stability.

In recent years, there has been a rise in the utilization of low-level laser therapy (LLLT) to treat numerous disorders and injuries. LLLT has been proven to be a safe and effective therapeutic option in clinical and histological trials demonstrating significant improvement in serious chronic disorders. Numerous studies also have exhibited LLLT's ability to modulate a variety of cellular reactions in non-photosynthetic cells. For example, laser therapy has been shown to stimulate the differentiation of satellite stem cells and improve wound healing and modulate chronic inflammation. A continually growing body of evidence suggests that laser therapy can alter cell bioenergetics. Consequently, LLLT can influence the functional biochemical properties intracellularly, culminating in an observable diverse clinical effect.

A recent study employing LLLT demonstrated an overall reduction in total circumferential measurement of the waist, hip, and thighs of −3.51 inches (p<0.001). It has been found that following the application of LLLT, adipocytes release 99% of their cellular content including fat. Following application of laser light at 635 nm, a transitory pore develops in the cellular wall of the adipocyte leading to its collapse. The intracellular fat is relatively fluid and, when the transitory pore occurs, will flow out of the cell into the interstitial space of adipose tissue. The interstitial space includes the connective tissue as wells as nerves, blood vessels, and lymphatics, among other substances. Because the fatty material within the cell is the predominate contributor to excessive fat mass, a laser-induced opening within the cell's protective barrier would advantageously provide a pathway for the fat to exit the cell and reduce the volume. Laser-provoked fat volume loss could restore proper adipocyte function thereby acting as an anti-diabetes mechanism.

In a separate study, the application of laser therapy at 635 nm demonstrated the reduction of TNF-α following the induction of a partial thickness burn in murine models. The reduction of TNF-α was found to be statistically significant (p<0.01) when compared to non-laser treated murine models.

It would be desirable to treat diabetes or prediabetes by decreasing a patient's intercellular fat and reducing a patient's TNF-α. It further would be desirable to treat diabetes without medications and their side effects. Therefore, an object of this invention is to provide a non-invasive method of treating diabetes with low-level laser therapy.

### SUMMARY OF THE INVENTION

This invention is a method of treating diabetes or prediabetes through the application of low-level laser therapy (LLLT). LLLT is applied externally through the skin of the patient to targeted areas of a of a patient's body. The targeted areas correspond to areas of the patient's body where there is the greatest accumulation of subcutaneous fat. Applying LLLT to the targeted areas reduces the intracellular fat and TNF-α levels.

To treat diabetes or prediabetes with LLLT, a patient must first be diagnosed with diabetes or prediabetes, preferably by blood draws that measure insulin, A1C, or glucose levels. After confirming a diagnosis of diabetes or prediabetes, low-level laser energy is applied to the targeted areas preferably with a 635 nm scanning laser of less than 1 watt of power. A manually scanned laser or mechanically scanned laser can be used. Low-level laser energy is applied daily, or more preferably three times a week, for at least two weeks and often ninety days. The amount of laser energy applied during each treatment will depend on the patient's diet, weight, and lifestyle. After the initial two weeks, follow-up blood work and physician visits monitor the patient's diabetes diagnosis. If diabetes is still present, low-level laser energy treatment continues, followed by further monitoring of blood work and physicians visits. Treatment times and frequency will be altered depending on the outcome of the prior treatment sessions. Treatments with low-level laser energy continue until the diabetes or prediabetes diagnosis is removed.

### BRIEF DESCRIPTION OF THE DRAWINGS

FIG. 1 is illustrates the application of low-level laser radiation.

FIG. 2 is a schematic illustration of normal fat cells.

FIG. 3 is a schematic illustration of fat cells after externally-applied low-level laser radiation.

FIG. 4 is a schematic illustration of a laser and optical arrangement for applying laser light according to the preferred embodiment of the present invention.

FIG. 5 is a perspective view of the first embodiment of a full-body laser scanner of the present invention.

FIG. 6 is top view of the first embodiment of the full-body scanner of the present invention.

FIG. 7 is bottom view of the first embodiment of the full-body laser scanner of the present invention.

FIG. 8 is a perspective view of the laser guidance system of the first embodiment of the full-body laser scanner of the present invention.

FIG. 9 is a side view of the laser guidance system of the first embodiment of the full-body laser scanner of the present invention.

FIG. 10 is a perspective view of the second embodiment of the full-body laser scanner of the present invention.

FIG. 11 is a perspective view of the third embodiment of the full-body laser scanner of the present invention.

### DETAILED DESCRIPTION OF THE INVENTION

The present invention is a method of treating a patient diagnosed with diabetes or prediabetes with low-level laser therapy. To treat diabetes or prediabetes with LLLT, a patient must first be diagnosed with diabetes or prediabetes. Preferably the diabetes or prediabetes diagnosis is confirmed by blood draws that measure insulin, A1C, or glucose levels. For example, a patient's fasting plasma glucose level can be measured, and a diagnosis of diabetes will be confirmed if the level is at or above 7.0 mmol/L. Prediabetes is confirmed if the patient's fasting plasma glucose level is between 5.6 mmol/L and 6.9 mmol/L. Alternatively, other generally accepted diabetes or prediabetes diagnosis methods may be used, as will be known by someone skilled in the art.

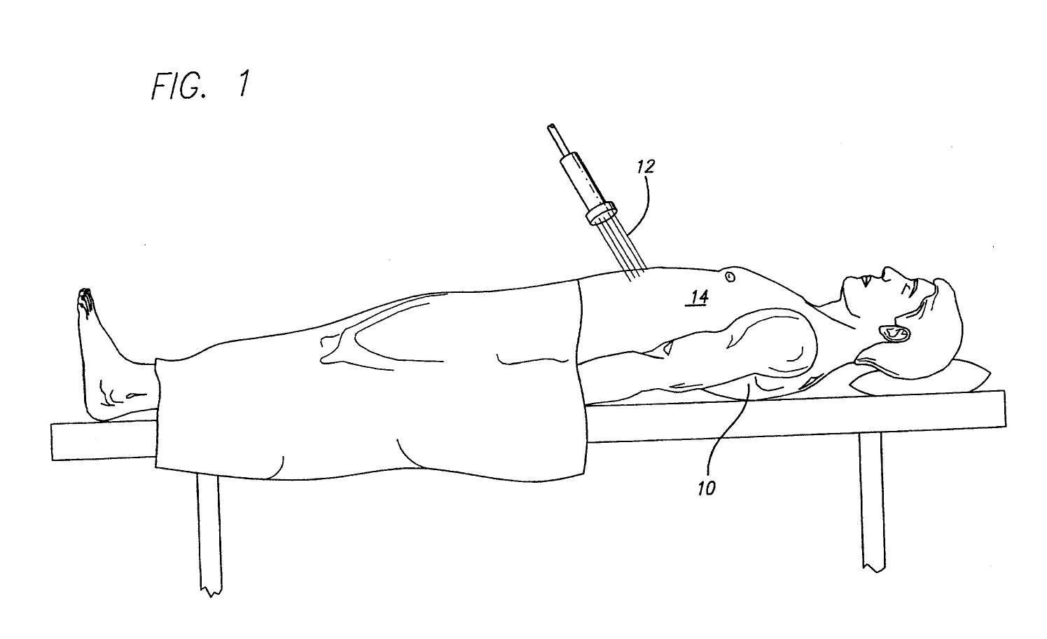

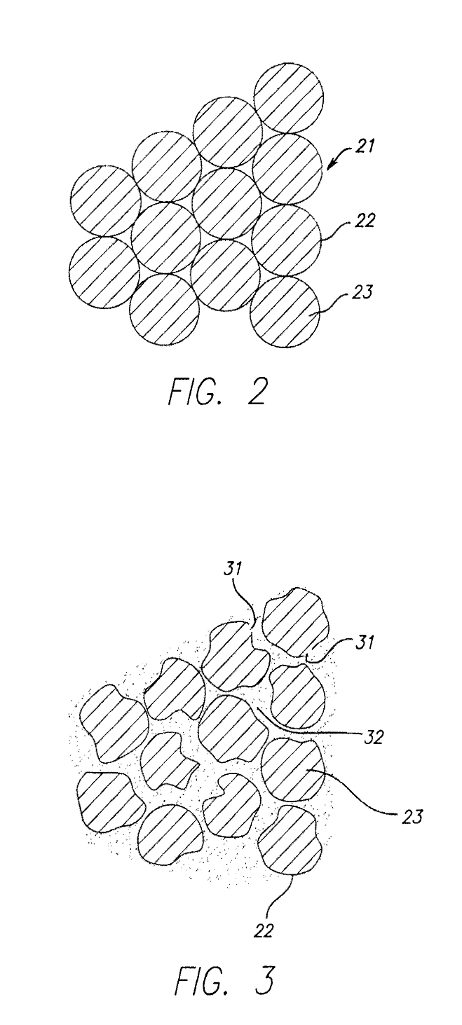

After confirming a diagnosis of diabetes or prediabetes, a therapeutic amount of laser energy to be applied is determined, and then the therapeutic amount of laser energy 12 is applied to targeted areas of the patient's body 10, as shown in FIG. 1. Preferably, the targeted areas of the patient's body are made up of adipocyte tissue below the skin 14 of the patient, as illustrated in FIG. 2. Sufficient laser energy is applied to the adipocyte tissue through the skin 14 to release at least a portion of the intracellular fat 23 into the interstitial space 32. FIG. 3 illustrates pores 31 in the cellular membrane 22 that have released intracellular fat 23 into the interstitial space 32. Upon cessation of the energy application, the pores 31 close and the cell membrane 22 returns to contiguity. The released intracellular fat and damaged fat cells are then removed from the body through the body's normal systems, such as metabolic, lymphatic, or excretory systems.

While the therapeutic amount of low-level laser energy applied should be sufficient to release at least a portion of the intracellular fat 23 into the interstitial space 32, the applied laser energy should not cause any immediate detectable temperature rise of the treated tissue in the targeted area or any macroscopically visible changes in tissue structure. Additionally, the low-level laser energy should not cause any sensation to be felt by the patient. The proper dose of low-level laser energy will penetrate the skin and be specific to the depth of the desired zone to be treated. Consequently, the treated and surrounding tissue will not be heated or damaged.

The preferred treatment time is about 20 minutes of laser energy application, three times a week preferably for two weeks. Alternatively, treatments can continue for 90 days or more. Alternatively, applying twenty minutes of laser energy can be repeated more or less frequently, such as daily treatments or treatments once or twice a week. Additionally, the length of time during which laser energy is applied can be increased or decreased depending on the patient's condition, weight, diet, lifestyle, thickness of the patient's skin, thickness of fatty tissue, and other biological factors peculiar to each patient. In certain situations, treatment times of less than one minute may be successful, and in others much longer treatment times will be successful. Those skilled in the art of low-level laser energy treatments will be able to determine and adjust the laser treatment time, treatment area, and laser intensity to accommodate biological factors peculiar to each patient. A laser practitioner may rely on his experience or may look for objective measurements to guide him when adjusting laser treatment time, treatment area, and laser intensity. For example, a person skilled in the art may use a patient's body mass index (BMI) as a guide for selecting an effective initial treatment protocol. A patient's BMI can be calculated by various methods known in the art. Based on the patient's BMI, the therapeutic dosage of low-level laser energy can be determined. A preferred therapeutic dosage is generally in the range of 0.03 J/cm²to 5.0 J/cm². For example, if a patient has a BMI that is between 15 and 24, then the treatment time and intensity are adjusted so that at least 15 Joules of laser energy over an area of 400 cm² is applied. If a patient has a BMI that is between 25 and 31, then the treatment time is adjusted so that 21 Joules of laser energy over an area of 400 cm² is applied. If a patient's BMI is over 31, then the treatment time is adjusted so that 30 Joules of laser energy over an area of 400 cm² is applied.

The laser energy can be applied to the patient using a variety of laser devices, such as a hand-held laser device, a wall-mounted laser device, or a stand-alone laser device, as described in more detail below. Preferably the laser energy is applied by a scanning laser. Alternatively, the laser energy can be applied by a therapist freely moving a non-scanning laser energy source over the targeted area. Alternatively, the laser energy can be emitted from a stationary source, such as an arm that emits laser energy that is attached to a wall or a stand, or a full-body scanning table or chamber.

Any individual adipocyte can become enlarged thus releasing deleterious bioactive substances promoting an insulin resistant state. Promoting insulin sensitivity within all adipocytes is preferable; therefore, whole body stimulation or multiple anatomical region stimulation of subcutaneous fat tissue along with visceral fat tissue is desired. A device which spans the entire treatment bed has the capacity to treat the entire body simultaneously or select anatomical regions to be treated in succession. The application of laser therapy can be applied to multiple anatomical regions of the body concurrently or separately.

Furthermore, the application of laser therapy can be coupled with mechanical stimulation including vibration either present on the device or on the treatment table. Mechanical stimulation can help promote lymphatic drainage which will help promote fatty material mobilization.

Preferably laser light visible to the human eye is used so that the area of application is easily determined. Most low-level laser treatments have proven to be effective at a single wavelength in the red region of the spectrum, between about 630 nm and about 670 nm. However, it has been shown that LLLT can be effective throughout the visible, near infrared and near ultraviolet regions. Laser diodes are currently available to cover only a limited part of the available spectrum, so other laser energy sources may be used. To obtain the maximum benefit it may be desirable to stimulate the patient at two or more different wavelengths. Persons skilled in the art will be aware that various laser energy sources are known in the art for use in low-level laser therapy. They include Helium-Neon lasers having a 632 nm wavelength and semiconductor diode lasers with a broad range of wavelengths between 405-1500 nm. The preferred laser is a semiconductor diode emitting laser light in the red range of the spectrum, having wavelengths of about 635 nm with maximum power of 20 mW. A preferred laser device that provides low-level energy includes the inventions described in U.S. Pat. Nos. 6,013,096 issued to Tucek and 6,746,473, issued to Tucek and Shanks.

A preferred embodiment is described as having a single laser energy source, but it will be appreciated that the invention may have two or more laser energy sources. The laser sources may be attached to each other in a laser assembly or individually attached to a support structure. While may LLLT regimen include ultraviolet or infrared laser light, it is advantageous to utilize at least one laser beam in the visible energy spectrum so that the operator can see the laser light as it impinges the patient's body and the area treated can be easily defined.

Laser energy also can be applied with a laser device that has one or more laser energy sources to accommodate different therapy regimens requiring diodes of different wattages. The preferred laser diodes use less than one watt of power each to simultaneously facilitate fat reduction, treat inflammation, treat pain, and reduce a patient's TNF-α. More preferably, the laser diodes have a maximum power of 20 mW. Diodes of various other wattages, however, may also be employed to achieve the desired laser energy for the given regimen. Low-level lasers are available commercially.

Another laser device particularly useful for this application is the stand-alone multi-laser scanner described in US Patent Publication 2006/0095099 invented by Shanks and Tucek. Additional technical details of the multi-laser scanner are disclosed in US Patent Publication 2006/0095099 and incorporated herein by reference. Another scanning laser device useful for this application is the laser scanner described in US Patent Publication 2006/0224218 invented by Tucek and Shanks. Additional technical details of the laser scanner are disclosed in US Patent Publication 2006/0224218 and incorporated herein by reference.

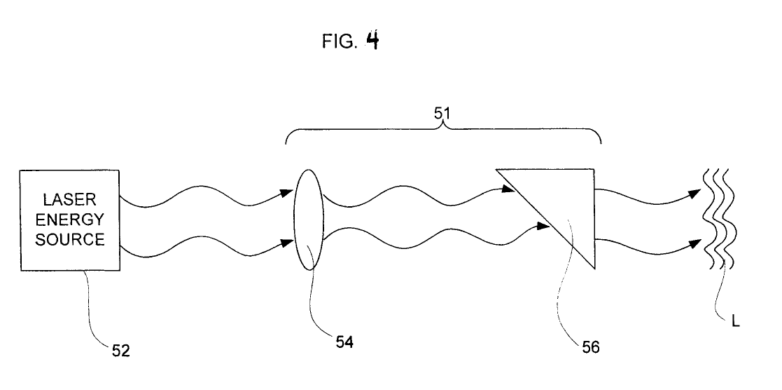

The laser device can optionally include optics for shaping the beam and creating beam spots, as described in U.S. Pat. No. 6,746,473 issued to Tucek and Shanks and incorporated herein by reference. In the preferred embodiment, laser energy is applied with a laser device capable of creating a linear spot shape. By using a linear beam spot, the number of times the laser light must be rastered is minimized or eliminated. FIG. 4 illustrates a laser device with optics for shaping the beam and creating a linear beam spot. As shown in FIG. 4, a laser device includes an optical arrangement 51 having a collimating lens 54 and a line generating prism 56 disposed in serial relation to the laser energy source 52. The collimating lens 54 and line generating prism 56 receive and transform the generated beam of laser light into a linear beam spot of laser light L. As an alternative, a suitable electrical or mechanical arrangement could be substituted for the optical arrangement to achieve a desired spot shape.

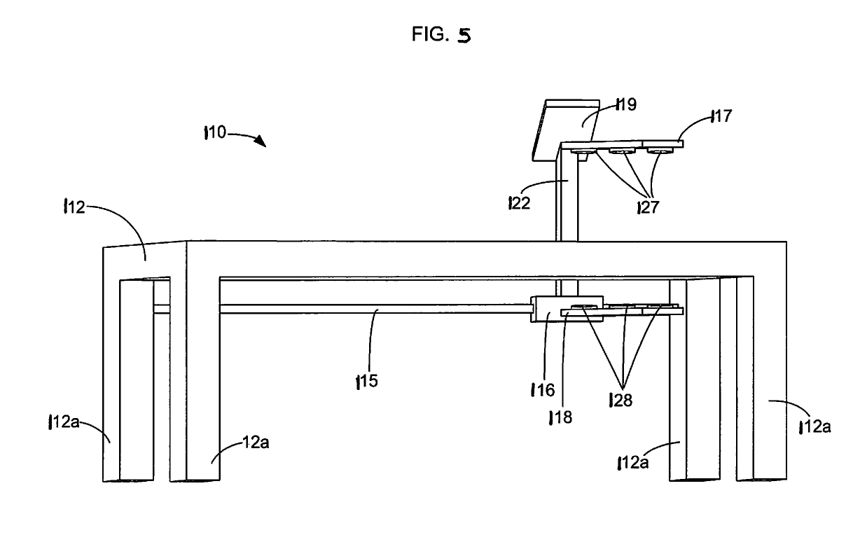

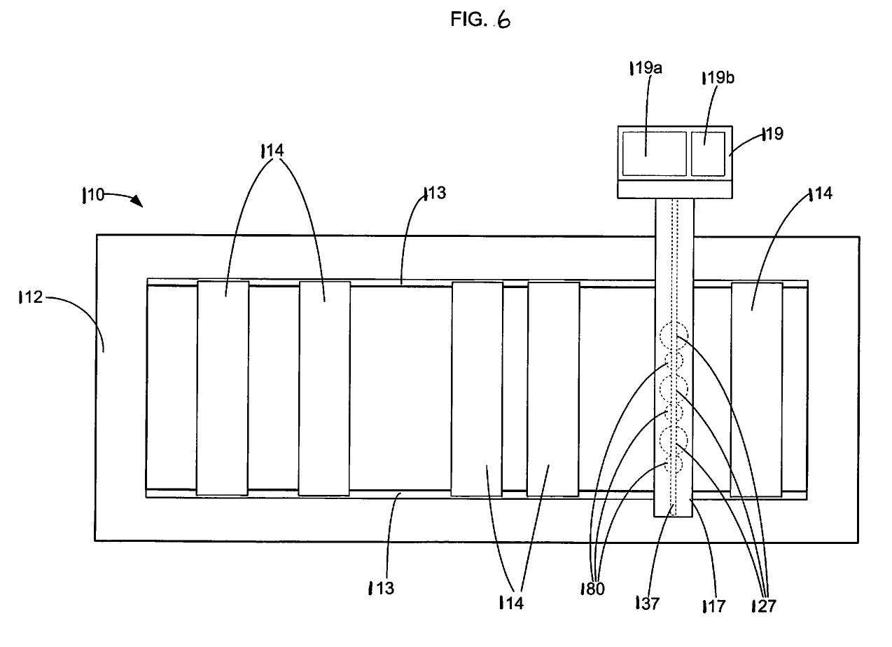

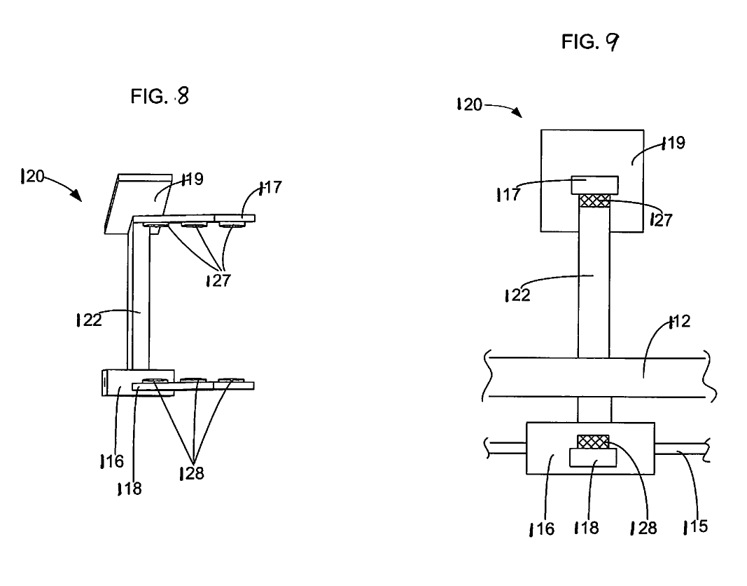

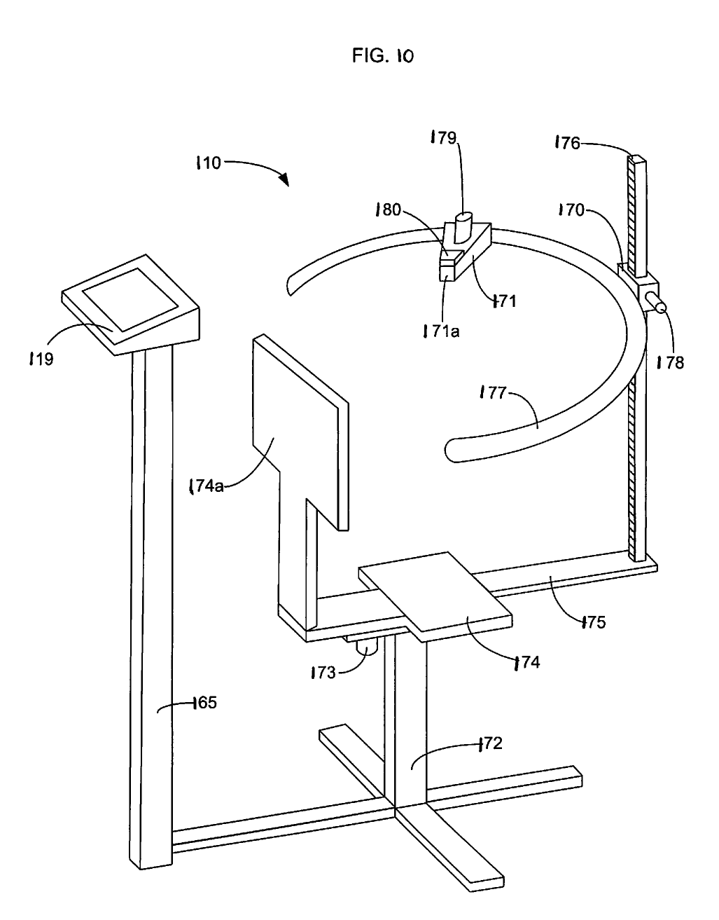

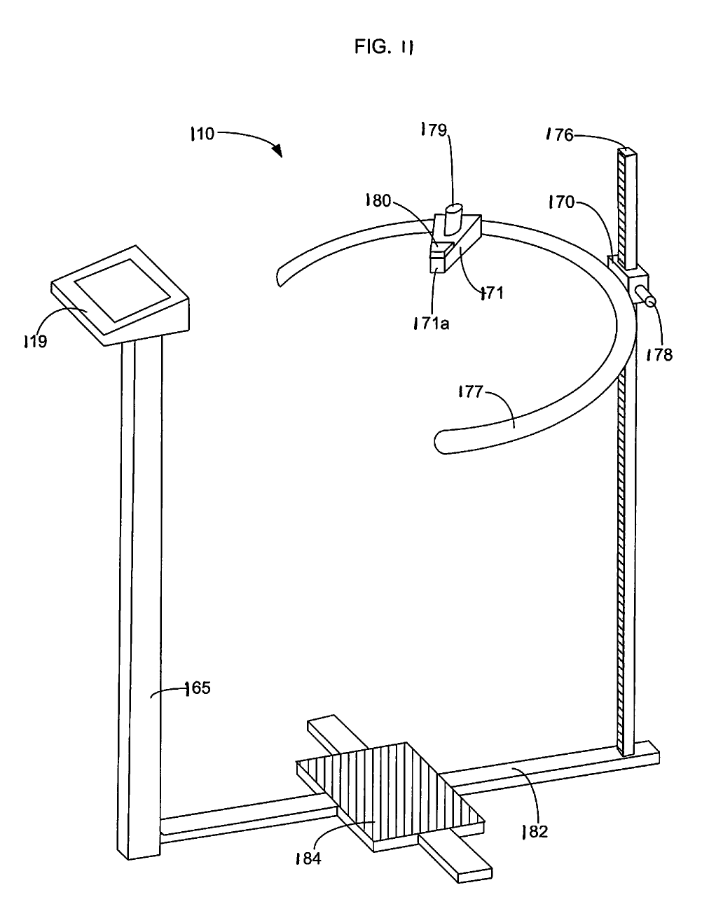

In the preferred embodiment, a full body laser scanner is used to deliver laser energy to the patient. A full body scanner 110 comprises a patient support, a laser guidance system, one or more laser devices, and a control center and control system. Full body scanner 110 additionally optionally includes feedback sensors. FIGS. 5-9 illustrate a first embodiment of scanner 110 where the patient support is a base 112 with a bed. FIG. 10 illustrates a second embodiment of scanner 110 where the patient support is a chair 174, and FIG. 11 illustrates a third embodiment of scanner 110 where the patient support is a platform 182.

Referring to FIGS. 5-9, the first and preferred embodiment of scanner 110 comprises a table or base 112 with a bed preferably comprised of movable slats 114 and a laser guidance system 120. Base 112 can be any type of table or housing for a bed as long as base 112 is capable of partially or substantially supporting the weight of a patient, a part of a patient, or any other subject requiring laser energy treatment. As shown in FIGS. 1-4, base 112 is a frame with four supporting legs 112a. Alternately, base 112 can comprise pedestals, corbels, shelf-supports, a wall, or any other support structure as is known in the art. The base or bed can also be adjustable such that a portion of it can be elevated and a portion not elevated, such that it can convert to a chair or lounge, or such that the entire bed is sloped.

The bed is positioned on base 112 to create a resting surface on which a patient can lay prone. As shown in FIGS. 5-9, the bed is a series of repositionable slats 114 resting in channels 113 formed in base 112. The base and movable slats permit a patient to be treated on one more sides without realignment. For example, when a patient lies on the slats 114 face up, his front is exposed above the slats 114 for laser treatment and his back is exposed for laser treatment through the moveable slats 114. Alternately, the bed can be any support structure capable of retaining a patient in the prone position that does not substantially interfere with the application of laser light at the treatment area resting on the bed. For example, the bed can be a transparent solid surface, a series of narrow transparent or non-transparent rods, or a taut mesh surface, as will be understood by someone skilled in the art. If the bed is comprised of supports that can be positioned and repositioned, preferably the supports, e.g. rods or slats, can be moved or translated to expose different regions on a patient depending on the treatment protocol. For example, a patient can lay prone on translating slats that can be positioned to expose the abdomen for abdominal laser treatments or positioned to expose thighs for laser treatments on the patient's thighs. The supports can be moved manually or a motor and control system can be operationally connected to the supports to automatically move them according to a given protocol. For this embodiment, the slats would not need to be transparent.

As shown in FIGS. 5-9, the laser guidance system of the first embodiment is a linear guidance system that comprises a carriage rail 115 and a carriage assembly 116. The carriage assembly 116 translates on carriage rail 115 and comprises an upper arm 117, a lower arm 118, and vertical stanchion 122. Lower arm 118 extends below the bed and supports a plurality of lower laser devices 128 that are directed toward the bed. Vertical stanchion 122 extends upwards from carriage rail 115 and connects to upper arm 117, which extends above the bed and supports a plurality of upper laser devices 127 that are directed toward the bed. A control system and control center 119 cooperates with carriage assembly 116 and can be mounted directly on stanchion 122, upper arm 117, or lower arm 118. Control system and center 119 operate a drive motor (not shown) housed within carriage assembly 116 to control the carriage assembly's movement on carriage rail 115. Control center 119 also individually operates the laser devices.

Carriage rail 115 and carriage assembly 116 can be any type of linear guidance system that facilitates linearly translating an object, such as a track roller linear guidance system. For example, carriage rail 115 can be a guideway with a track on which the carriage assembly 116 is supported and rolls. Alternatively, carriage rail 115 can be a belt or conveyer system along which carriage assembly 116 translates. Any type of linear guidance system can be substituted, as is known in the art.

Carriage assembly 116 translates along carriage rail 115 and can house a battery, control circuitry, and any other components necessary to facilitate the carriage's movement along the carriage rail. Carriage assembly 116 also can house any components necessary for operating any of the plurality of lasers. Carriage assembly 116 also cooperates with control center 119, which can be physically, electrically, or wirelessly connected to carriage assembly 116.

Upper arm 117 is connected to carriage assembly 116 by vertical stanchion 122 so that upper arm 117 translates as carriage assembly 116 translates. Upper arm 117 can be fixed to stanchion 122 or it can be moveably attached such that the height of upper arm 117 is adjustable. Alternately, upper arm 117 can connect directly to or be integral with carriage assembly 116. Vertical stanchion 122 extends upwards from carriage assembly 116 and transparent bed 114 as shown in FIGS. 1-4. Upper arm 117 then extends over and across the width of transparent bed 114 as shown in particular in FIG. 6. The portion of upper arm 117 that extends over and across transparent bed 114 supports one or more upper laser devices 127. Each upper laser device 127 is oriented so that laser energy is directed toward transparent bed 114. Preferably, upper arm 117 also includes a horizontal alignment guide 137 on which each of the upper laser devices 127 can be repositioned and a vertical alignment system (not shown) for individually raising and lowering each of the upper laser devices 127. For example, each upper laser device 127 also can be lowered toward the patient or raised toward upper arm 117 without interfering with the operation of the upper laser devices 127. Preferably, each of the upper laser devices 127 are controlled by the control system and control center and moved automatically with horizontal and vertical movement drive motors (not shown).

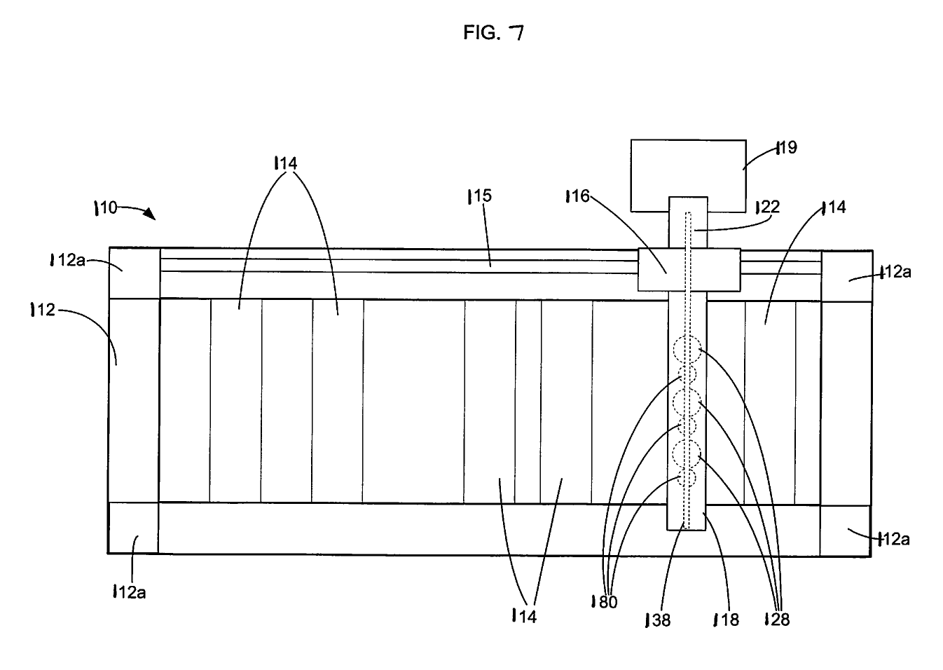

Lower arm 118 is integral with or connected either directly or indirectly to carriage assembly 116 so that lower arm 118 translates as carriage assembly 116 translates. Lower arm 118 extends below and across the width of bed 114 as shown in particular in FIG. 7. Lower arm 118 can also be height adjustable and can extend downwards from carriage assembly 116 to create more clearance between transparent bed 114 and lower arm 118 if needed. The portion of lower arm 118 that extends below and across transparent bed 114 supports one or more lower laser devices 128. Each lower laser device 128 is oriented so that laser energy is directed toward transparent bed 114. Preferably, lower arm 118 also includes a horizontal alignment guide 138 on which each of the lower laser devices 128 can be repositioned and a vertical alignment system (not shown) for individually raising and lowering each of the lower laser devices 128. For example, each lower laser device 128 also can be raised toward the patient or lowered toward lower arm 118 without interfering with the operation of the lower laser devices 128. Preferably, each of the lower laser devices 128 are controlled by the control center and moved automatically with horizontal and vertical movement drive motors (not shown).

Upper laser devices 127 and lower laser device 128 can be any laser device that provides low-level energy. A laser device that provides low-level energy is known in the art as a cold laser, such as the inventions described in U.S. Pat. No. 6,013,096 issued to Tucek and U.S. Pat. No. 6,746,473, issued to Tucek and Shanks. The preferred laser is a semiconductor diode emitting laser light in the red range of the spectrum, having wavelengths of about 635 nm. Other lasers known in the art for use in low-level laser therapy include Helium-Neon lasers having a 632 nm wavelength and semiconductor diode lasers with a broad range of wavelengths between 405-1500 nm. Diode lasers at 625 nm, 633 nm, 670 nm and 1064 nm (infrared) have been shown to work with varying degrees of success. The laser device may have one or more laser energy sources. Different therapy regimens require diodes of different wattages. The preferred laser diodes use less than one watt of power each to simultaneously facilitate fat reduction, treat post-operative inflammation, and treat post-operative pain. Diodes of various other wattages may also be employed to achieve the desired laser energy for the given regimen. Low-level lasers are available commercially.

The laser device can optionally include optics for shaping the beam to create desired spot shapes, as described in U.S. Pat. No. 6,746,473 issued to Tucek and Shanks. In the preferred embodiment, laser energy is applied with a laser device capable of creating a linear spot shape. By using a line of laser light, the number of times the laser light must be scanned back and forth across the targeted area is minimized. FIG. 4 illustrates a laser device with optics for shaping the beam and creating a linear shape. As shown in FIG. 4, a laser device includes an optical arrangement 51 having a collimating lens 54 and a line generating prism 56 disposed in serial relation to the laser energy source 52. The collimating lens 54 and line generating prism 56 receive and transform the generated beam of laser light into a line of laser light L. As an alternative, a suitable electrical or mechanical arrangement or combination thereof could be substituted for or combined with the optical arrangement to achieve a desired spot shape.

Upper and lower laser devices 127 and 128 can also be laser scanning devices such as the inventions described in U.S. Published Patent Application 2006/0095099 belonging to Shanks and Tucek, which is incorporated herein by reference. By using laser scanning devices, the line generating prism can be operated to scan laser light in any pattern, as described in the U.S. Published Patent Application 2006/0095099.

Upper arm 117 and lower arm 118 also support feedback sensors 180 either separate from or as part of the upper and lower laser devices 127 and 128. The sensors 180 are preferably ultrasonic proximity sensors that will give vertical distance feedback regarding the distance between laser devices 127 and 128 and the patient's body. Along with the vertical distance feedback from feedback sensors 180, linear movement encoders track the horizontal movement of the laser devices. The combination permits three-dimensional movement of the laser devices to maximize the areas being treated. Moreover, by tracking the vertical distance of the laser devices in relation to the patient being irradiated with laser energy, an active scan can be accomplished. The laser devices 127 and 128 can be positioned as the patient is being treated with laser energy.

The laser guidance system 120, the laser devices 127 and 128, and the feedback sensors 180 can all be operated with a control system, which is partially or substantially located in control center 119. Control center 119 also preferably incorporates a display 119a and operator input device 119b such as a touch screen or a separate keyboard and screen. Using control center 119, the operator can select and input information relative to the treatment protocol, operation of the full-body scanner, and patient details. The control system is comprised of various discrete circuits, as is known in the art. For example, the control system includes control circuitry for operating the laser guidance system 120, the laser devices 127 and 128, and the sensors 180. In a further form, the control system includes a microprocessor programmed to operate in various modes. While the invention is not limited to any particular programmed operation mode, by way of example the following modes of operation are available:
1. The carriage assembly 116 is programmed to move linearly through a series of fixed regions and dwell for a pre-set period at each region for laser application by the upper and lower laser devices 127, 128 at the region or at a specific targeted area. The regions or specific targeted areas may be input by an operator or user to align with particular positions on the body that require treatment.
2. The vertical displacement of the upper and lower laser devices 127, 128 is programmed to move vertically through a series of fixed heights and dwell for a pre-set period at each region for laser application by the upper and lower laser devices 127, 128 at the region or at a specific targeted area. The regions or specific targeted areas may be input by an operator or user to align with particular positions on the body that require treatment.
3. The upper and lower laser devices 127, 128 are programmed to move through a series of fixed regions and dwell for a pre-set period at each region. The regions may be input by a user to align with particular positions on the body that require treatment.
4. The wavelength of applied laser energy is periodically changed by changing the operating laser diode in one or more of the upper and lower laser devices 127, 128 during a repetitive scan. This allows stimulation of the targeted area at multiple wavelengths.
5. The focal position of the beam shaping optics of one or more of the upper or lower laser devices 127, 128 is changed to generate smaller or large spot sizes on the targeted area.
6. The laser power of one or more of the upper and lower laser devices is varied.

FIG. 10 illustrates a second and preferred embodiment of scanner 110 where the patient support is a base 172 with a seat 174. As shown, the full-body laser scanner 110 also comprises one or more laser devices 171, a control center 119, and a laser guidance system that comprises a vertical stanchion 176, an orbital guide ring 177, and a carriage assembly 170. The second embodiment of scanner 110 can also optionally include feedback sensors 180. Vertical stanchion 176 is preferably connected to base 172 either directly or with an arm 175 that extends away from seat 174. Alternately, vertical stanchion 176 can be a standalone structure. Preferably, vertical stanchion 176 is in a fixed position such that it does not move when, for example, seat 174 moves or carriage assembly 170 moves.

Base 172 supports seat 174, and seat 174 can be any type of seat capable of supporting a patient who is sitting. The seat preferably accommodates a patient sitting facing stanchion 176, facing away from stanchion 176, or facing to one side or other. For example, a patient can sit on seat 174 shown in FIG. 10 with his back resting on the seat back 174a, with his chest resting on the seat back 174a, or with either of his shoulders or arms resting on the seat back 174a. Seat 174 also preferably pivots on base 172 and can be rotated manually or optionally rotated with a center mass rotational drive motor 173. The base and seat permit a patient to be treated on one more sides without realignment. For example, when a patient sits with his right shoulder on the seat back 174a, his front and back are exposed for laser treatment.

The laser guidance system of the second embodiment is an orbital guidance system comprising an orbital guide ring 177 and carriage assembly 170 as shown in FIG. 10. While only one orbital guide ring 177 and carriage assembly 170 is shown, any number of orbital guide rings and carriage assemblies can be used in any combination. For example, one carriage assembly may support two or more orbital guide rings. Likewise, multiple orbital guide rings may each be supported by a separate carriage assembly. The carriage assembly 170 translates on vertical stanchion 176 and comprises vertical movement drive motor 178. Motor 178 can be any drive motor capable of moving a structure up and down a vertical support or stanchion, as is known in the art. If multiple orbital guidance systems are desired, each can have a vertical stanchion or all of the orbital guidance systems can be supported by a single vertical stanchion, or any combination thereof. Carriage assembly 170 can house a battery, control circuitry, and any other components necessary to facilitate the carriage's movement along the stanchion 176. Carriage assembly 116 also can house any components necessary for operating any of lasers 171. Carriage assembly 170 also cooperates with control center 119, which can be physically, electrically, or wirelessly connected to carriage assembly 170.

Orbital guide ring 177 is connected to carriage assembly 170, and is directed towards seat 174 such that it forms a partial or full ring around a patient sitting on seat 174. Orbital guide ring 177 can be any type of substantially circular rails or guides capable of supporting a translating object, as is known in the art. Orbital guide ring 177 is either integral with carriage assembly 170 or otherwise rigidly secured to carriage assembly such that the movement of laser devices 171 along ring 177 are not impeded and such that as carriage assembly translates along stanchion 176, orbital guide ring 177 translates vertically as well.

Orbital guide ring 177 further supports one or more laser devices 171 that are directed inward toward the center of orbital guide ring 177. Preferably, laser devices 171 are aligned along the radius of orbital guide ring 177 such that the laser output location 171a is also aligned along the radius of orbital guide ring 177. Laser devices 171 can translate along guide ring 177 and are preferably driven by an orbital drive motor 179 connected to each of laser device 171. Motor 179 can be any drive motor capable of moving a structure along an orbital guide ring, as is known in the art. Preferably, each of the laser devices 171 are controlled by the control system as well as each of the orbital drive motors 179.

Laser devices 171 can be any laser device that provides low-level energy. For further information regarding laser devices 171, see the discussion above for upper laser devices 127 and lower laser devices 128. Also see the discussion above regarding beam-shaping optics and laser scanning devices, both of which apply to laser devices 171 as well.

Each laser device 171 can also support a feedback sensor 180 either separate from laser device 171 or as part of laser devices 171. As shown in FIG. 10, sensor 180 is positioned outside of laser device 171. Alternatively, it can be positioned within the same housing as laser device 171. The sensors 180 are preferably ultrasonic proximity sensors that will give distance feedback for the laser devices 127 and 128. Along with the distance feedback from feedback sensors 180, movement encoders track the movement of the laser devices. Additionally movement encoders track the vertical movement of the carriage assembly 170 and accordingly the orbital ring 177 and laser devices 171. The combination permits three-dimensional movement of the laser devices to maximize the areas being treated. Moreover, by tracking the distance of the laser devices in relation to the patient being eradiated with laser energy, an active scan can be accomplished. The laser devices can be positioned as the patient is being treated with laser energy. Ultrasonic proximity sensors and movement encoders are well known in the art.

In the laser guidance system shown in FIG. 10, each of the laser devices 171, and each of the feedback sensors 180 can all be operated with the control system, which is partially or substantially located in control center 119. Control center 119 is preferably mounted on a separate support 165 or alternatively is mounted on vertical stanchion 176, carriage assembly 170, base 172, or seat 174. As described above with respect to the first embodiment, control center 119 also preferably incorporates a display and operator input device, such as a touch screen, where the operator can select and input information relative to the treatment protocol, operation of the full-body scanner, and patient details. The control system is comprised of various discrete circuits, as is known in the art. For example, the control system includes control circuitry for operating the laser guidance system, each of the laser devices 171, and each of the sensors 180. In a further form, the control system includes a microprocessor programmed to operate in various modes as described above with respect to the first embodiment of this invention.

FIG. 11 illustrates a third embodiment of scanner 110 where the patient support is a base 182 and platform 184. As shown, the full-body laser scanner 110 comprises a base 182 with a platform 184, one or more laser devices 171, a control center 119, and an orbital laser guidance system that comprises a vertical stanchion 176, an orbital guide ring 177, and a carriage assembly 170. The third embodiment of scanner 110 can also optionally include feedback sensors 180 and movement encoders.

Base 182 supports platform 184, and platform 184 can be any type of structure capable of supporting a patient who is standing. The platform preferably accommodates a patient standing facing stanchion 176, facing away from stanchion 176, or facing to one side or other. Platform 184 also preferably pivots on base 182 and can be rotated manually or optionally rotated with a center mass rotational drive motor (not shown). The base and platform permit a patient to be treated on one more sides without realignment. For example, when a patient stands on the platform, his front and back are exposed for laser treatment.

The carriage assembly 170, orbital guide ring 177, laser devices 171, optional feedback sensors 180 and movement encoders, drive motors 178 and 179, and control system and control center 119 for the third embodiment of full body scanner 110 are described above with respect to the second embodiment of full body scanner 110.

Generally, to treat a patient's body with the full-body laser scanner 110, the patient is aligned on the patient support, and the laser devices and the feedback sensors are driven about the patient's body. With the control panel, an operator can implement any preprogrammed treatment protocols or can manually control the carriage's movement and the lasers. The carriage assembly and laser devices translate according to the chosen protocol or manual instructions. Additionally, the laser devices apply laser energy according to the chosen protocol or manual instructions, the feedback sensors give feedback regarding the distance between the laser devices, and the contours of the patient's body and the movement encoders track the location or position of the laser devices. Tracked movements of the laser devices along with distance measurements provided by the feedback sensors provide the data for creating a map of the patient's contours and identifying targeted areas of the body for laser treatment.

Using the first embodiment of the full-body laser scanner as an example, the patient lies, preferably on his back, on the bed of slats 114. With the control center 119, an operator chooses any preprogrammed treatment protocols or can manually control the carriage assembly's 116 movement and lasers devices 127, 128. The carriage assembly 116 translates from one end of the base 112 and bed to the other in either one continuous translation or with several back and forth translations of various amounts. While the carriage assembly 116 translates, the upper arm 117 translates accordingly and upper laser devices 127 apply laser energy to the surfaces of the patient's body that are directed upwards. Simultaneously, the lower arm 118 translates accordingly and lower laser devices 128 apply laser energy to the surfaces of the patient's body that are directed downwards. Using the sensors 180, the full-body laser scanner 110 can collect reflected or backscattered ultrasound waves and process them along with information regarding the location of the laser devices 127, 128 to map the contours of the patient's body and identify targeted areas of the body for laser treatment. Alternatively, the operator can manually identify targeted areas of the body for laser treatment. If desired, upper laser devices 127 can be raised or lowered to apply laser energy to the surfaces of the patient's body that are directed upwards, and lower laser devices 128 can be raised or lowered to apply laser energy to the surfaces of the patient's body that are directed downwards. Next, low-level laser energy can be applied to the targeted areas of the patient's body to treat diabetes and prediabetes. Laser energy can also be applied according to treatment protocols for other laser applications.

The full body scanner 110 and any laser device can further include means to vibrate the patient before, during, or after laser treatment. Mechanical stimulation can be achieved with, for example, a vibrating belt or reciprocating plunger against the skin, a vibrating table that patient lies on, a vibrator attached to or integral with a laser probe or diode head, or a vibrating probe that extends from a hand-held laser device.

After the initial two weeks or an initial period of time up to 90 days, follow-up blood work and physician visits monitor the patient's diabetes or prediabetes diagnosis. Additionally, a person skilled in the art will also monitor changes in the patient's biological factors. Preferably the diabetes or prediabetes diagnosis is monitored with blood draws that measure insulin, A1C, or glucose levels. Alternatively, other generally accepted diabetes diagnosis monitoring methods may be used, as will be known by someone skilled in the art. If diabetes or prediabetes is still present, low-level laser energy treatments continue, followed by further monitoring of blood work, monitoring of biological factors, and physicians visits.

Treatment times and the frequency of treatment sessions will be altered depending on the outcome of the prior treatment sessions, on any changes to a patient's biological factors, and on the results from any tests monitoring the diabetes diagnosis and condition. For example, if the monitoring of blood work suggests that a patient's diabetes condition is only moderately responding to the LLLT, then the treatment time or treatment frequency may need to be increased. Alternatively, if the blood work suggests that a patient's condition is improving rapidly, treatment times or frequencies can be reduced. Someone skilled in the art will be able to determining whether to increase or decrease treatment times or the therapeutic amount of laser energy and whether to increase or decrease the frequency of treatment sessions. Treatments with low-level laser energy continue until the diabetes or prediabetes diagnosis is removed.

### EXAMPLE 1

A patient is diagnosed with diabetes after two fasting plasma glucose measurements at or above 7.0 mmol/L. The targeted area for apply laser energy is the patient's abdomen and hips. The patient's abdomen and each of the patient's hips are treated with laser energy using a 635 nm semiconductor diode laser with maximum power of 20 mW. The laser energy is applied for 20 minutes in a back-and-forth sweeping motion across the first hip without touching the patient, and the laser energy is applied for 20 minutes in a back-an-forth sweeping motion across the second hip without touching the patient. Then the laser energy is applied for 20 minutes in a back-and-forth sweeping motion across the abdomen without touching the patient. The laser energy is applied so that there is no temperature rise or macroscopically visible change in the targeted areas. Treatment is repeated for the abdomen and each hip at a rate of three treatment sessions of each week over a total of 90 days The patient's fasting plasma glucose is again measured, and a moderate decline in fasting plasma glucose levels is observed. The protocol is repeated for another 90 days with increases to laser application time and frequency. The laser energy is applied for 30 minutes in a back-and-forth sweeping motion across the first hip without touching the patient, and the laser energy is applied for 30 minutes in a back-and-forth sweeping motion across the second hip without touching the patient. Then the laser energy is applied for 30 minutes in a back-and-forth sweeping motion across the abdomen without touching the patient. The laser energy is applied so that there is no temperature rise or macroscopically visible change in the targeted areas. Treatment is repeated for the abdomen and each hip at a rate of four treatment sessions each week over a total of 90 days. The patient's fasting plasma glucose is again measured, and significant improvement is observed. The diagnoses of diabetes is removed.

### EXAMPLE 2

A patient is diagnosed with prediabetes after two fasting plasma glucose measurements between 5.6 mmol/L and 6.9 mmol/L. The targeted area for apply laser energy is the patient's abdomen. The patient's abdomen is treated with laser energy using a 635 nm semiconductor diode laser with maximum power of 20 mW. The laser energy is applied for 30 minutes in a back-and-forth sweeping motion across the abdomen without touching the patient. The laser energy is applied so that there is no temperature rise or macroscopically visible change in the targeted area. Treatment is repeated daily for the abdomen daily over a two week period. The patient's fasting plasma glucose is again measured, and a moderate decline in fasting plasma glucose levels is observed. The protocol is repeated without any changes for another two weeks. The patient's fasting plasma glucose is again measured, and significant improvement is observed. The diagnoses of prediabetes is removed.

### EXAMPLE 3

A patient is diagnosed with diabetes after two fasting plasma glucose measurements at or above 7.0 mmol/L. The targeted area for applying laser energy is the patient's abdomen. The patient's abdomen is treated with laser energy using a 635 nm semiconductor diode laser with maximum power of 20 mW. The laser energy is applied for 30 minutes in a back-and-forth sweeping motion across the abdomen without touching the patient. The laser energy is applied so that there is no temperature rise or macroscopically visible change in the targeted areas. Treatment is repeated for the abdomen daily over a two week period. The patient's fasting plasma glucose is again measured, and a rapid decline in fasting plasma glucose levels is observed. The protocol is repeated for another two weeks with decreases to laser application time and frequency. The laser energy is applied for 20 minutes in a back-and-forth sweeping motion across the abdomen without touching the patient. The laser energy is applied so that there is no temperature rise or macroscopically visible change in the targeted areas. Treatment is repeated for the abdomen five more times over a two week period. The patient's fasting plasma glucose is again measured, and significant improvement is observed. The diagnoses of diabetes is removed.

### EXAMPLE 4

A patient with a BMI of 26 is diagnosed with diabetes after two fasting plasma glucose measurements at or above 7.0 mmol/L. The targeted area for apply laser energy is the patient's abdomen. The patient's abdomen and each of the patient's hips are treated with laser energy, using a 635 nm semiconductor diode laser with maximum power of 20 mW. The laser energy is applied in a back-and-forth sweeping motion across the abdomen without touching the patient for a length of time that delivers 21 Joules over an area of 400 cm². The laser energy is applied so that there is no temperature rise or macroscopically visible change in the targeted areas. Treatment is repeated for the abdomen daily over a two week period. The patient's fasting plasma glucose is again measured, and a rapid decline in fasting plasma glucose levels is observed. Additionally, the patient's BMI has decreased to 24. The protocol is repeated for another two weeks with decreases to laser application time and frequency. The laser energy is applied in a back-and-forth sweeping motion across the abdomen without touching the patient for a length of time that delivers 15 Joules over an area of 400 cm². The laser energy is applied so that there is no temperature rise or macroscopically visible change in the targeted areas. Treatment is repeated for the abdomen five more times over a two week period. The patient's fasting plasma glucose is again measured, and significant improvement is observed. The diagnoses of diabetes is removed.

### EXAMPLE 5

A patient is diagnosed with prediabetes after two fasting plasma glucose measurements between 5.6 mmol/L and 6.9 mmol/L. The targeted area for apply laser energy is the patient's abdomen, and the patient is treated with a full body scanner 110. The patient lays face up on the slats 114 of the bed of the first embodiment of the full-body scanner 110. The carriage assembly translates to position the upper arm 117 over the patient's abdomen. The patient's abdomen is then treated with laser energy using a 635 nm semiconductor diode laser with maximum power of 20 mW. While the patient's abdomen is treated with laser energy, the patient's abdomen is vibrated by applying a hand-held oscillating deep massage device to the patient's abdomen skin. The laser energy is applied for 30 minutes at the abdomen without touching the patient. The laser energy is applied so that there is no temperature rise or macroscopically visible change in the targeted area. Treatment is repeated daily for the abdomen daily over a two week period. The patient's fasting plasma glucose is again measured, and a moderate decline in fasting plasma glucose levels is observed. The protocol is repeated without any changes for another two weeks. The patient's fasting plasma glucose is again measured, and significant improvement is observed. The diagnosis of prediabetes is removed.

While there has been illustrated and described what is at present considered to be a preferred embodiment of the present invention, it will be understood by those skilled in the art that various changes and modifications may be made, and equivalents may be substituted for elements thereof without departing from the true scope of the invention. Therefore, it is intended that this invention not be limited to the particular embodiment disclosed as the best mode contemplated for carrying out the invention, but that the invention will include all embodiments falling within the scope of the appended claims.

We claim:

1. A method of treating diabetes or prediabetes in a patient comprising:
a) diagnosing diabetes or prediabetes;
b) determining from a patient's biological factors an appropriate therapeutic amount of laser energy to apply; and
c) applying the determined therapeutic amount of laser energy to the patient.

2. The method of claim 1 wherein the laser energy has a wavelength in the red range.

3. The method of claim 1 wherein the laser energy has a wavelength of about 635 nm.

4. The method of claim 1 wherein the determined therapeutic amount of laser energy is at least 0.03 joules/cm².

5. The method of claim 1 wherein the patient's biological factors include a body mass index between 15-24 and the amount of laser energy applied is at least 0.03 joules/cm².

6. The method of claim 1 wherein the patient's biological factors include a body mass index between 25-31 and the amount of laser energy applied is at least 0.05 joules/cm².

7. The method of claim 1 wherein the patient's biological factors include a body mass index higher than 31 and the amount of laser energy applied is at least 0.07 joules/cm².

8. The method of claim 1 wherein the determined therapeutic amount of laser energy is applied to the patient at least two times over at least a two week time period.

9. The method of claim 1 wherein the determined therapeutic amount of laser energy is applied six times over at least a two week time period.

10. The method of claim 1 wherein the determined therapeutic amount of laser energy is applied daily for at least two weeks.

11. The method of claim 8 further comprising retesting the patient for diabetes or prediabetes after two weeks of applying the determined therapeutic amounts of laser energy to the patient.

12. The method of claim 11 further comprising:
a) determining from a patient's biological factors an appropriate second therapeutic amount of laser energy to apply; and
b) applying the second determined therapeutic amount of laser energy to the patient.

13. The method of claim 12 wherein the determined therapeutic amount of laser energy is applied at least two times over a two week period.

14. The method of claim 1 wherein diabetes or prediabetes is diagnosed by measuring a patient's insulin level.

15. The method of claim 1 wherein diabetes or prediabetes is diagnosed by measuring a patient's glucose level.

16. The method of claim 1 wherein diabetes or prediabetes is diagnosed by measuring a patient's A1C level.

17. The method of claim 1 wherein the laser energy is applied by a scanning laser device.

18. The method of claim 1 further comprising applying mechanical stimulation to the patient.

19. A method of treating diabetes or prediabetes in a patient comprising:
a) drawing a first blood sample from the patient;
b) measuring the level of glucose in the first blood sample;
c) applying one or more times over at least a two week time period at least 0.03 joules/cm² of laser energy to the patient using a laser device;
d) drawing a second blood sample from a patient;
e) measuring the level of glucose in the second blood sample;
f) comparing the level of glucose in the second blood sample to the level of glucose in the first blood sample; and
g) adjusting the therapeutic amount of laser energy to apply in light of the change in glucose level.

20. The method of claim 19 further comprising:
a) determining the patient's body mass index before applying a therapeutic amount of laser energy
b) determining the patient's body mass index after applying a therapeutic amount of laser energy one or more times over at least a two week time period; and
c) further adjusting the therapeutic amount of laser energy to apply in light of the change in body mass index.
